(19) United States
(12) Patent Application Publication
(10) Pub. No.: US 2023/0073795 A1
(43) Pub. Date: Mar. 9, 2023

(54) Customized Tracheocutaneous Fistula and Tracheostomy Plug

(71) Applicant: THE REGENTS OF THE UNIVERSITY OF MICHIGAN, Ann Arbor, MI (US)

(72) Inventors: Kyle VanKoevering, Ann Arbor, MI (US); Glenn E. Green, Gregory, MI (US); Keith A. Casper, Ann Arbor, MI (US); Brittny N. Tillman, Southlake, TX (US)

(21) Appl. No.: 17/984,434

(22) Filed: Nov. 10, 2022

Related U.S. Application Data
(62) Division of application No. 16/123,503, filed on Sep. 6, 2018.

(51) Int. Cl. A61M 16/04; B33Y 80/00; B33Y 50/00; B29C 33/38

(52) U.S. Cl. CPC A61M 16/0465; B33Y 80/00; B33Y 50/00; B29C 33/3842; B29L 2031/753

(57) ABSTRACT

A customized tracheocutaneous fistula and tracheostomy plug having a face plate and a body customized to the anatomy of a patient is disclosed. The face plate has a face plate diameter, and the body has a first end connected to the face plate and a second end, a shoulder connected at the second end, and a tapered tip extending from the shoulder. The body has a body length extending from the first end to the second end and a shoulder diameter. The face plate diameter and the shoulder diameter are each greater than a measured minimum stoma diameter, and the body length is equal or substantially equal to a measured stoma length. Also, a mold-based method and a direct method of manufacturing the plug.

### FIELD OF THE DISCLOSURE

This disclosure relates generally to a plug for a tracheocutaneous fistula or tracheostomy and, more specifically, to a customized plug created for a patient's unique anatomy. A method of manufacturing the plug is also disclosed.

### BACKGROUND

Tracheotomy is a surgical procedure wherein an incision is made along the trachea to open the airway. The resulting tracheostomy (i.e., stoma or hole) along the neck can serve as an independent airway and/or as a site for a tracheostomy tube placement to enable respiration. Approximately 100,000 tracheotomies are performed in the United States annually.

Patients having a tracheostomy experience many challenges that can significantly impact their quality of life, such as a persistent air leak, a weak voice, and difficulty with showering or bathing. A rigid tracheostomy tube often disrupts swallowing function, can be uncomfortable, and can cause significant skin breakdown. Furthermore, the typical tracheostomy tube is unsightly and can cause significant social isolation due to the stigma associated with a tracheostomy.

These challenges are particularly pronounced for patients who need to protect their tracheostomy or whose tracheostomy is no longer used but remains incompletely closed. For example, some patients who undergo a tracheotomy have conditions that require only intermittent access to the trachea. In these patients, once fully healed, the tracheostomy tube remains in place around the clock but is capped or plugged throughout the day. For such patients, the tracheostomy tube becomes essentially a bulky, unsightly placeholder during the day, maintaining the access to the trachea, though not routinely needed. With a rigid or semi-rigid capped tracheostomy tube in place, however, the cap frequently dislodges, and secretions leak around the tracheostomy tube. A tie secures the tube around the neck to prevent it from being coughed out, and further increases the discomfort and risk for skin breakdown. Even if the tracheostomy tube is removed, occasionally the stoma does not heal properly. These patients develop tracheocutaneous fistulae after tracheostomy tube removal for a variety of reasons. In some patients, surgical closure of a fistula is felt to be an unsafe or undesirable solution.

Unfortunately, the plugging and capping devices currently on the market suffer from key shortcomings and are suboptimal for many patients. The existing devices are not customizable to individual patient anatomy and are often ill-fitting as a result. The existing devices may require surgical placement. Additionally, the existing devices may not be easily removed in case of emergency.

### SUMMARY

Embodiments within the scope of the present disclosure are directed to a customized tracheocutaneous fistula and tracheostomy plug that uses a novel technique to ensure a perfect fit with individual patient anatomy and can be self-retained within the tracheostomy. The disclosed plug is a simple, single-piece design. The disclosed plug is extremely low profile and does not require neck ties to be secured in place. The disclosed plug fully seals the stoma, regardless of unique patient anatomy, which results in reduced skin irritation, air leaks, and secretions through the stoma. The disclosed plug is formed from a flexible material that improves comfort, swallowing function, and voice strength. Further, the disclosed plug can be easily removed by the patient, allowing immediate access to the trachea if needed. This significantly improves safety in case of a medical emergency.

One aspect of the disclosure is directed to a customized trachoeocutaneous fistula and tracheostomy plug having a face plate and a body. The face plate has a face plate diameter. The body has a first end connected to the face plate and a second end, a shoulder connected at the second end, and a tapered tip extending from the shoulder. The tapered tip allows easy placement of the plug through an existing stoma. The body has a body length extending from the first end to the second end and a shoulder diameter. The face plate diameter and the shoulder diameter are each greater than a measured minimum stoma diameter, and the body length is equal or substantially equal to a measured stoma length. As a result, the plug fits securely in the stoma of the patient from who the measured minimum stoma diameter and the measured stoma length were taken. Specifically, the face plate keeps the plug from being pulled into the trachea of the patient, thereby preventing aspiration and choking. The face plate diameter is significantly larger than the minimum stoma diameter, and in some cases may be at least three times greater than the measured minimum stoma diameter, to provide a large margin of error that virtually eliminates any possibility that the plug might be pulled or aspirated into the trachea. Meanwhile, the shoulder keeps the plug from being expelled outward from the stoma (i.e. with coughing, swallowing, speaking or neck movement), thereby securely sealing the stoma with a self-retaining design. Because the body length is equal or substantially equal to the measured stoma length, the face plate and the shoulder keep the plug from sliding back and forth within the stoma, thereby reducing irritation and discomfort.

In some arrangements, the plug may have a body geometry between the first end and the second end, and the body geometry may be complementary to a measured stoma surface geometry. For example, in some instances, the body geometry may have a curvature in one direction in order to be complementary to a curvature in the stoma. As another example, in some instances, the body geometry may be angled relative to the face plate as opposed to meeting the face plate at a 90 degree angle in order to accommodate the angle of the stoma. As yet another example, ridges, bumps, or asymmetries within the stoma may be accommodated. The ability to alter the body geometry to complement a measured stoma surface geometry ensures that the plug fits and seals the stoma in a way that a traditional, non-custom plug cannot. This feature is particularly important for patients having unique or complicated stoma geometry.

In some arrangements, the face plate of the plug may have a face plate shape, and the face plate shape may be complementary to a measured neck surface geometry. For example, in some instances, the face plate shape may be asymmetrical and/or three-dimensionally contoured in order to better accommodate a skin contour on the measured neck surface geometry. The ability to alter the face plate shape to complement a measured neck surface geometry ensures that the face plate will fit comfortably against a patient's neck and will, in some cases, be less noticeable than a face plate that protrudes from the natural folds of the neck. Additional efforts may be made to help the face plate blend in with a patient's neck or, alternately, to include decorative features chosen by the user. For example, the face plate may be colored to match a measured neck surface color. The face plate may be colored darker in color for a patient having a darker skin tone and lighter in color for a patient having a fairer skin tone. Additionally, the face plate may include a decorative pattern or include a decorative topography. The decorative pattern or topography may help the face plate to blend into the patient's neck, such as by including typical skin features such as moles, skin tone variations, and the like. Alternately, the decorative pattern or topography may be chosen by the user as a decorative feature, operating as a conversational piece or personal statement in the same manner as a tattoo, jewelry, or colored bands on orthodontic braces.

In some arrangements, the plug may comprise a semi-rigid material. Allowing the material to have some degree of flexibility can improve the comfort of wearing the plug for the patient. Specifically, the plug may be formed from medical grade silicone. An example of an acceptable medical grade high-durometer silicone that may be used is RTV 45 produced by Factor II, Inc. in Lakeside, Ariz.

In some arrangements, the plug may comprise a cannula from an outer surface of the face plate to an outer surface of the tapered tip. This enables the plug to be used to access the trachea and/or for airflow. The plug may further comprise a cap that is attachable to the cannula.

In some arrangements, at least one of the measured minimum stoma diameter, the measured stoma length, the measured neck surface geometry, and the measured stoma surface geometry is taken from at least one of hand-collected detailed measurements, a 3D reformatted CT scan, a 3D reformatted MRI scan, and a surface 3D scan. The hand-collected detailed measurements may be collected from a patient using flexible and rigid endoscopes, cotton tip swabs, rulers, and calipers, among other methods and devices. Additional imaging and/or measurement techniques may also be used in order to collect the various measurements.

Another aspect of the disclosure is directed to a mold-based method of manufacturing a tracheocutaneous fistula and tracheostomy plug, such as the plug discussed above. The method includes measuring a minimum stoma diameter and measuring a stoma length. The method further includes creating a virtual 3D mold for the plug having a virtual negative space for a face plate having a face plate diameter greater than the minimum stoma diameter, and a body having a first end connected to the first plate and a second end, a shoulder connected at the second end, and a tapered tip extending from the shoulder, wherein the body has a length extending from the first end to the second end that is equal or substantially equal to the stoma length and a shoulder diameter that is greater than the minimum stoma diameter. The virtual 3D mold may be created using an FDA cleared 3D modeling software such as MIMICS Innovation Suite by Materialise ™ from Leuven, Belgium or another 3D modeling program. The method further includes manufacturing a physical mold having a physical negative space equivalent to the virtual negative space of the virtual 3D mold. Finally, the method includes filling the physical negative space of the physical mold with a mold material to form the plug and allowing the mold material forming the plug to cure.

In the method, as described above, the minimum stoma diameter and the stoma length may each be determined based on at least one of hand-collected detailed anatomic measurements, a 3D reformatted CT, a 3D reformatted MRI, and a surface 3D scan. As described above, the hand-collected detailed measurements may be collected from a patient using flexible and rigid endoscopes, cotton tip swabs, rulers, and calipers, among other methods and devices. Additional imaging and/or measurement techniques may also be used in order to collected the various measurements.

The physical mold is manufactured to comply with FDA regulatory constraints. The physical mold may comprise a medical grade photopolymer. Specifically, the physical mold may be formed from an FDA cleared class I medical grade photopolymer. Manufacturing of the physical mold may include printing the mold initially using an additive manufacturing technique and then post-processing the physical mold after the initial creation of the physical mold. The mold material used to fill the mold may be a medical grade silicone. As described above, an example of an acceptable medical grade high-durometer silicone that may be used is RTV 45 produced by Factor II, Inc. in Lakeside, Ariz.

The method may include measuring a stoma surface geometry, wherein the virtual negative space includes a body geometry between the first end and the second end that is complementary to the stoma surface geometry. Similarly, the method may include measuring a neck surface geometry, wherein the virtual negative space includes a face plate geometry complementary to the neck surface geometry. The stoma surface geometry or the neck surface geometry may be determined based on at least one of a 3D reformatted CT, a 3D reformatted MRI, and a surface 3D scan.

Yet another aspect of the disclosure is directed to a direct method of manufacturing a tracheocutaneous fistula and tracheostomy plug, such as the one described above. The method includes measuring a minimum stoma diameter and measuring a stoma length. The method further includes creating a virtual 3D plug model, the virtual 3D plug model having a face plate having a face plate diameter greater than the minimum stoma diameter, and a body having a first end connected to the first plate and a second end, a shoulder connected at the second end, and a tapered tip extending from the shoulder, wherein the body has a length extending from the first end to the second end that is equal or substantially equal to the stoma length and a shoulder diameter that is greater than the minimum stoma diameter. The method further includes manufacturing the plug from the virtual 3D plug model using additive manufacturing.

The method may further include measuring a stoma surface geometry and measuring a neck surface geometry, wherein the virtual 3D plug model further comprises a body geometry between the first end and the second end that is complementary to the stoma surface geometry and a face plate geometry complementary to the neck surface geometry.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a cross-sectional view of a head and neck of a patient having a stoma with a standard tracheostomy tube of the prior art placed in the stoma;

FIG. 2 is a cross-sectional view of a head and neck of a patient having a stoma with the plug of the present disclosure placed in the stoma;

FIG. 3A is an isometric view of a plug of the present disclosure customized to fit a relatively small stoma resulting from a fistula with an asymmetric face plate customized to fit a skin contour;

FIG. 3B is a front view of the plug of FIG. 3A;

FIG. 3C is a side view of the plug of FIGS. 3A and 3B;

FIG. 3D is a back view of the plug of FIGS. 3A-3C;

FIG. 3E is a top view of the plug of FIGS. 3A-3D;

FIG. 3F is a bottom view of the plug of FIGS. 3A-3F;

FIG. 4A is an isometric view of a plug of the present disclosure customized to fit a relatively standard stoma;

FIG. 4B is a front view of the plug of FIG. 4A;

FIG. 4C is a side view of the plug of FIGS. 4A and 4B;

FIG. 4D is a back view of the plug of FIGS. 4A-4C;

FIG. 4E is a top view of the plug of FIGS. 4A-4D;

FIG. 4F is a bottom view of the plug of FIGS. 4A-4F;

FIG. 5A is an isometric view of a plug of the present disclosure customized to fit a relatively long stoma, such as the type of stoma common in obese patients;

FIG. 5B is a front view of the plug of FIG. 5A;

FIG. 5C is a side view of the plug of FIGS. 5A and 5B;

FIG. 5D is a back view of the plug of FIGS. 5A-5C;

FIG. 5E is a top view of the plug of FIGS. 5A-5D;

FIG. 5F is a bottom view of the plug of FIGS. 5A-5F;

FIG. 6A is an isometric view of a plug of the present disclosure having a cannula and a cap;

FIG. 6B is a front view of the plug of FIG. 6A;

FIG. 6C is a side view of the plug of FIGS. 6A and 6B;

FIG. 6D is a back view of the plug of FIGS. 6A-6C;

FIG. 7 is a schematic diagram of a mold-based method of manufacturing a plug of the present disclosure;

FIG. 8 is a custom mold based on the anatomy of a patient having a negative space that can be filled to create a plug of the present disclosure;

FIG. 9 is the custom mold of FIG. 8 filled with a medical grade silicone to create the plug;

FIG. 10 is the plug created from the mold shown in FIGS. 8 and 9; and

FIG. 11 is a schematic diagram of a direct method of manufacturing a plug of the present disclosure.

### DETAILED DESCRIPTION

The following detailed description is directed to a plug for a tracheocutaneous fistula or tracheostomy, and more specifically, to a customized plug created for a patient's unique anatomy using 3D printing and medical grade silicone molding. Related methods of manufacture are also disclosed.

For purposes of this specification and the appended claims, the term “diameter” is not understood or intended to imply a measurement of a fully circular or perfectly circular shape. Rather, the term “diameter” is understood to include any length across a circular, oval, ovoid, rounded, oblong, or irregularly shaped object. The elements for which a “diameter” is referenced herein may or may not be perfectly or fully circular. For example, a cross-section of a stoma may have a roughly circular but specifically irregular shape. Thus, the “minimum stoma diameter” referenced herein would simply be the shortest distance across an irregularly shaped cross-section of the stoma.

For purposes of this specification and the appended claims, the term “substantially equal” means that the percent difference between two measured quantities is less than or equal to 15%.

As used in this specification and the appended claims, the singular forms “a”, “an”, and “the” include plural references unless the context clearly dictates otherwise.

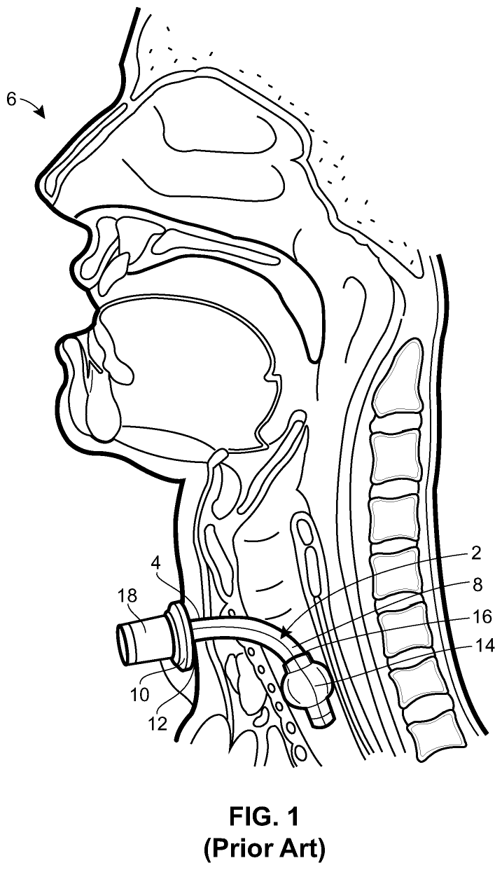

Referring to the figures in detail, FIG. 1 shows a traditional tracheostomy tube 2 placed in the stoma 4 of a patient 6. The tracheostomy tube 2 has a minimally flexible cylindrical cannula 8 having a standard flange 10 on an external side 12 and an inflatable balloon 14 on an internal side 16. The cannula 8 is selectively closed with a cap 18. The traditional tracheostomy tube 2 is not customized to the length or surface geometry of the stoma 4 of the patient 6. As a result, the cylindrical cannula 8 can slide within the stoma 4, causing irritation and discomfort. The flange 10 is also not customized to the anatomy of the patient 6 and may therefore rub against and protrude outward from the neck of the patient 6.

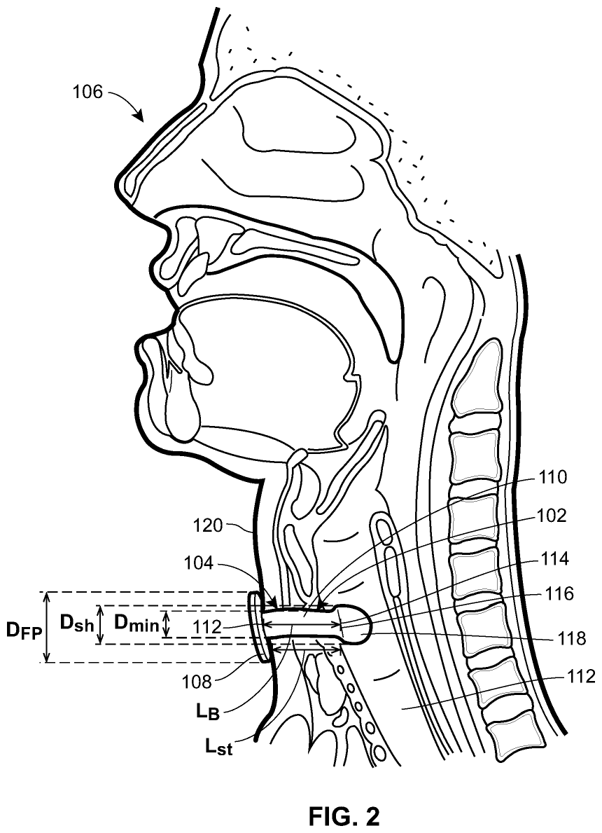

In contrast, FIG. 2 shows a customized tracheocutaneous fistula and tracheostomy plug 102 of the present disclosure placed in the stoma 104 of a patient 106. The plug 102 includes a customized face plate 108 and a body 110. The plug 102 may be formed of a single unitary component comprising the customized face plate 108 and body 110. The body 110 has a first end 112 connected to the face plate 108 and a second end 114, a shoulder 116 connected at the second end 114, and a tapered tip 118 extending from the shoulder 116. The face plate 108 and body 110 of the plug 102 may be made from a semi-rigid material, such as medical grade silicone. This ensures that the plug 102 has enough flexibility to be comfortable while still being firm enough to be functional.

As shown in FIG. 2, the plug 102 fits securely within the stoma 104 of the patient 106. In particular, the body 110 has a body length L_{B} extending from the first end 112 to the second end 114. The body length L_{B} is equal or substantially equal to a measured stoma length L_{St}. The face plate 108 has a face plate diameter D_{FP} and the shoulder 116 has a shoulder diameter D_{Sh}. The face plate diameter D_{FP} and the shoulder diameter D_{Sh} are each greater than a measured minimum stoma diameter Dₘᵢₙ. That is, the face plate 108 and the shoulder 116 are each too wide to fit through the narrowest portion of the stoma 104. For some plugs 102, the face plate diameter D_{FP} is at least three times greater than the measured minimum stoma diameter Dₘᵢₙ to virtually guarantee that the plug cannot move into the trachea of the patient 106. Moreover, the body 110 is prevented from moving within the stoma 104 because the body length L_{B} between the face plate 108 and the shoulder 116 is equal or substantially equal to the measured stoma length L_{St} such that the face plate 108 and shoulder 116 are flush against the neck 120 of the patient 106 and the trachea 122 of the patient 106 respectively. The result of these customized measurements is that the plug 102 fits the patient 106 better, resulting in improved comfort and functionality.

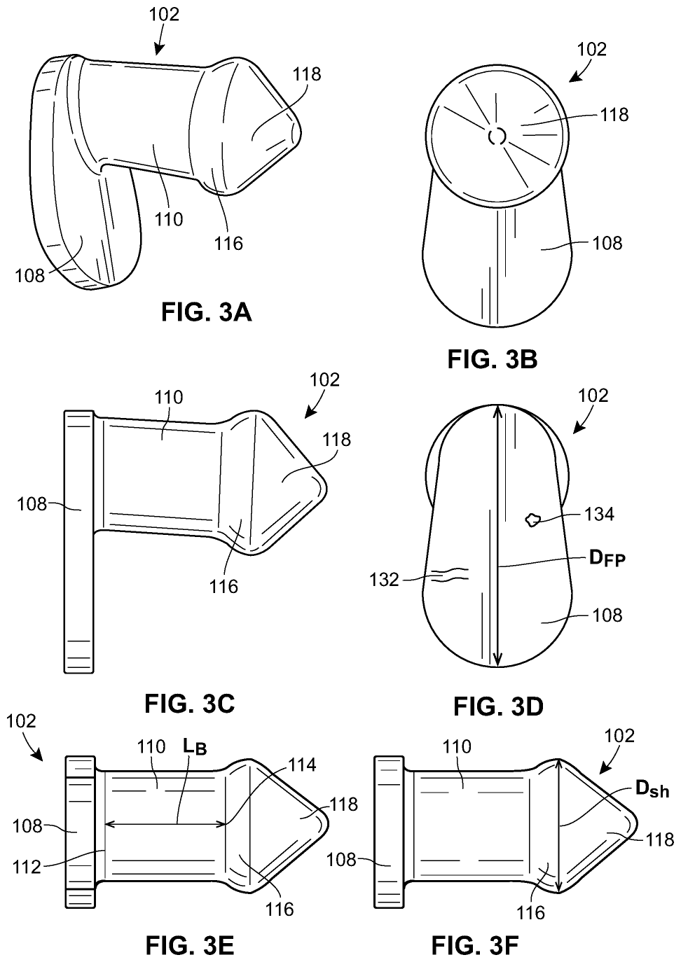

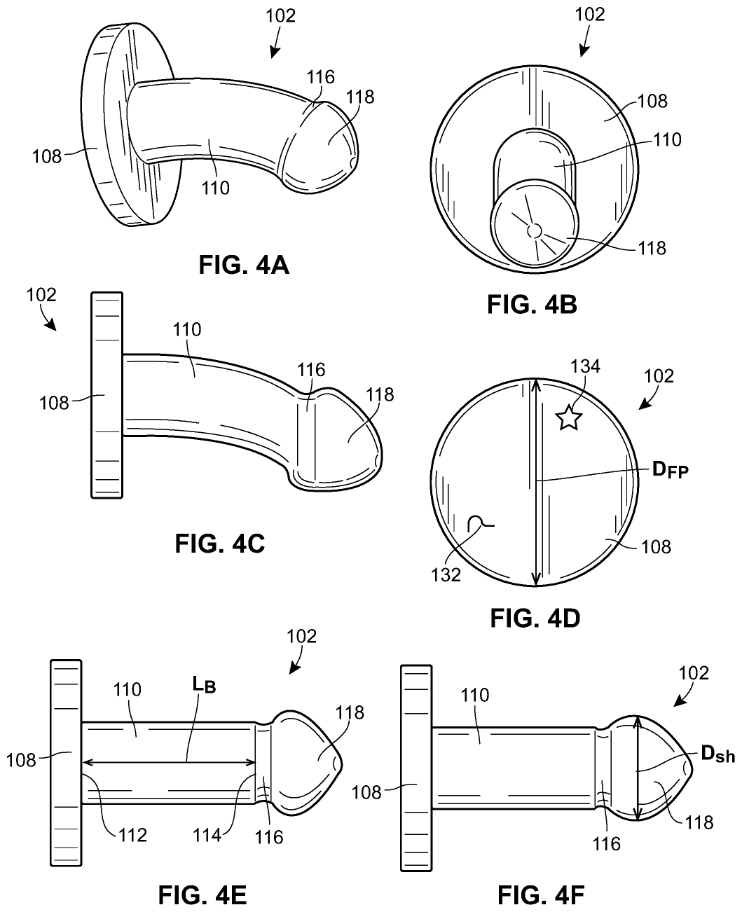

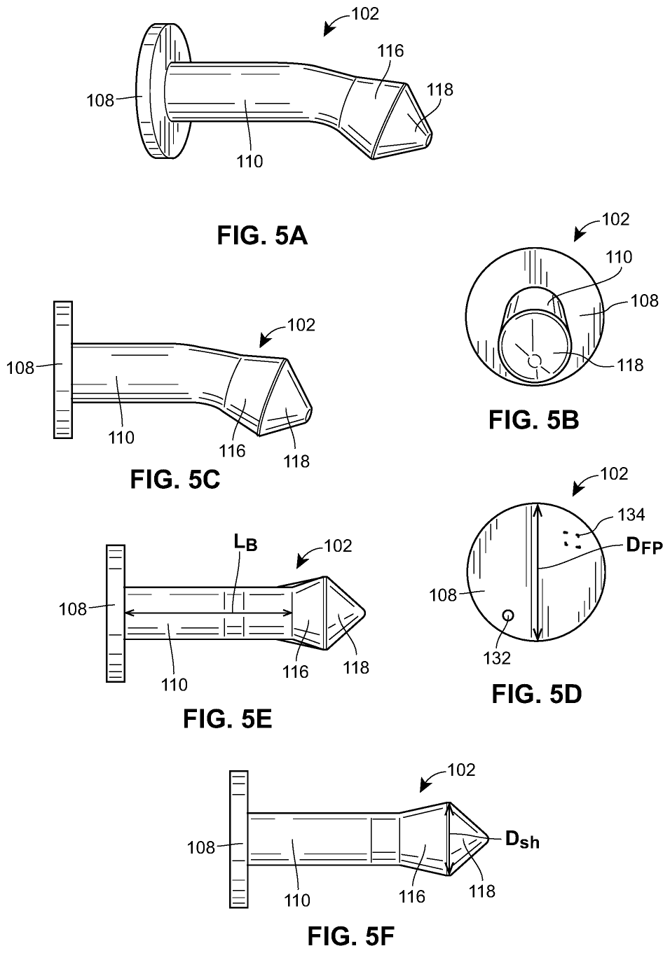

As shown in FIGS. 3A-5F, the body length L_{B} of the body 110 of the plug 102 may vary significantly depending on the anatomy of the patient 106. For example, the plug 102 shown in FIGS. 3A-3F has a shorter body length L_{B} than the plug 102 shown in FIGS. 4A-4F, and the plug 102 shown in FIGS. 5A-5F has the greatest length of all. The body length L_{B} may depend on the medical cause of the stoma 104. For example, a stoma 104 created by a fistula may be smaller than a stoma 104 created surgically for purposes of inserting a tracheostomy tube. The body length L_{B} may also affected by medical conditions, such as obesity, that the patient 106 has. The measured minimum stoma diameter Dₘᵢₙ may also be impacted by the medical cause of the stoma 104 and medical conditions of the patient 106. The measured minimum stoma diameter Dₘᵢₙ, in turn, affects the face plate diameter D_{FP} and shoulder diameter D_{Sh} of the plug 102. The plug 102 depicted in FIGS. 3A-3F might be more typical of a plug 102 for a patient having a stoma 104 created by a fistula, whereas the plug 102 depicted in FIGS. 5A-5F might be more typical of a plug 102 for an obese patient 106. However, the anatomy of each patient 106 will also result in some natural variations in the size of a stoma 104. Each plug 102 will have a customized relationship between the body length L_{B}, face plate diameter D_{FP}, and shoulder diameter D_{Sh}.

As also shown in FIGS. 3A — 5F, additional features of the plug 102 may be customized. The body 110 may have a body geometry between the first end 112 and the second end 114 that is complementary to a measured surface geometry of the stoma 104. As shown in FIGS. 3A-3F, the plug 102 may curve downward along the body length L_{B}. Alternately, the plug 102 may curve upward or in any direction. The plug 102 may have a relatively uniform width or may vary in width to complement the measured surface geometry along the body length L_{B}. The angle at which the body 110 meets the face plate 108 may be altered. The tapered tip 118 may be more or less rounded, may extend from the shoulder 116 at a variety of angles, and may vary in length and width.

Other portions of the plug 102 apart from the body 110 may also be customized. The face plate 108 may have a face plate shape that is complementary to a measured neck surface geometry. For example, as shown in FIGS. 3A-3F, the face plate 108 may be asymmetrical to accommodate a skin contour of the neck of the patient 106. The face plate 108 may additionally have a curved topography or may be rounded and eccentric in shape in order to better complement the measured neck surface geometry. Additionally, the face plate 108 may be colored to match a measured neck surface color. For example, the face plate 108 may have a flesh tone that is the same tone as the skin tone of the neck of the patient 106. Alternately, or in addition, the face plate may include a decorative pattern 134 or a decorative topography 132. In some instances, the decorative pattern 134 or decorative topography 132 may help the face plate 108 blend in with the neck 120 of the patient 106 by including, for example, a portion of a birth mark or freckles naturally occurring on the neck or a raised surface mimicking the natural folds and curvatures of the neck 120 of the patient 106. In other instances, the decorative pattern 134 or decorative topography 132 may be ornamental, such as by being a favorite color of the patient or including a meaningful image or icon (such as a favorite sports team mascot or a religious symbol). The decorative pattern 134 or decorative topography 132 of the face plate 108 may help to ease the stigma associated with having a stoma by either blending in so as to make the plug 102 less noticeable or by being a chosen point of interest and a conversation starter.

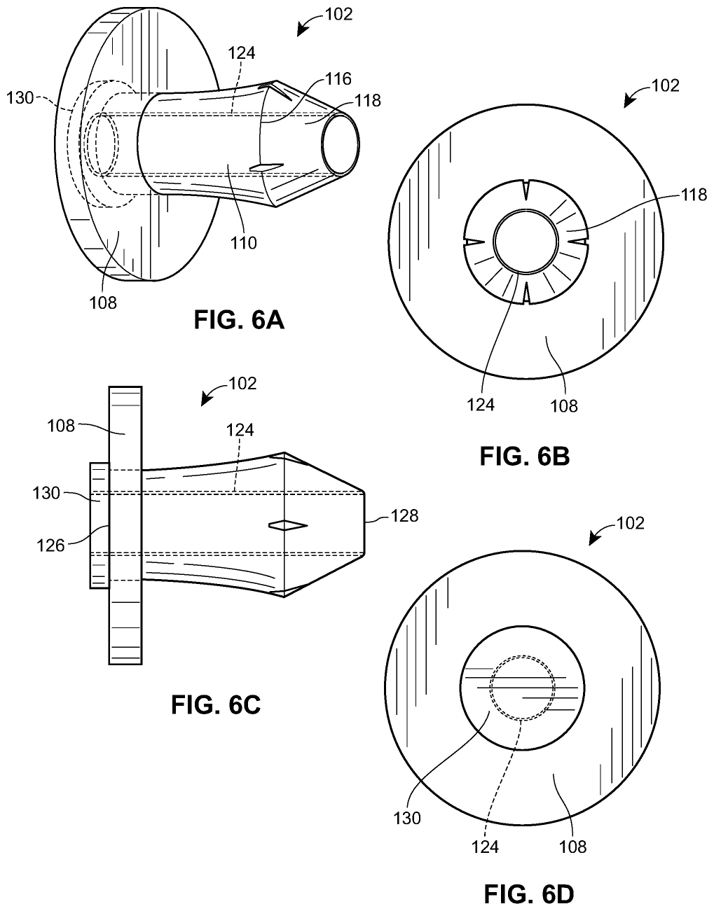

As shown in FIGS. 6A-6D, the plug 102 may include a cannula 124 extending from an outer surface 126 of the face plate 18 to an outer surface 128 of the tapered tip 118. The cannula 124 may be surrounded by the body 110 of the plug 102 in a manner that still allows customization and excellent fit. A cap 130 may be provided in or at the face plate 108 to allow selective access to the cannula 124. Like the face plate 108, the cap 130 may be designed to complement the measured neck surface geometry. The cap 130 may also be colored to match a neck surface color and/or may include a decorative pattern or decorative topography.

The various measurements taken from a patient 106 discussed above (measured stoma length L_{St}, measured minimum stoma diameter Dₘᵢₙ, measured surface geometry of the stoma 104, and measured neck surface geometry) may be collected in a number of different ways. For example, these measurements may be hand-collected detailed anatomic measurements determined by using flexible and rigid endoscopes, cotton tip swabs, rulers, and/or calipers. More advanced imaging techniques and computerized measuring techniques may also be used. For example, the measurements may be collected from a 3D reformatted CT, a 3D reformatted MRI, or a surface 3D scan.

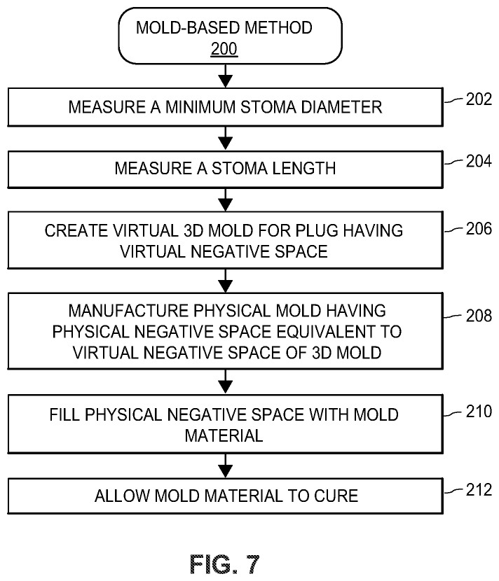

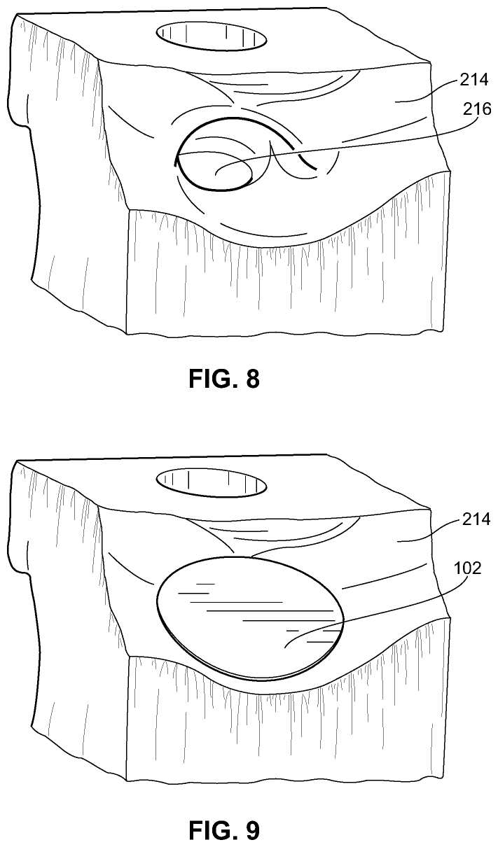

FIGS. 7-10 depict the process of creating a plug 102 via a mold-based method 200 of manufacturing. As shown in FIG. 7, the mold-based method 200 comprises, at box 202, measuring a minimum stoma diameter. At box 204, the method 200 comprises measuring a stoma length. At box 206, the method 200 comprises creating a virtual 3D mold for the plug having a virtual negative space for a face plate having a face plate diameter greater than the minimum stoma diameter, and a body having a first end connected to the first plate and a second end, a shoulder connected at the second end, and a tapered tip extending from the shoulder, wherein the body has a length extending from the first end to the second end that is equal or substantially equal to the stoma length and a shoulder diameter that is greater than the minimum stoma diameter. At box 208, the method 200 comprises manufacturing a physical mold having a physical negative space equivalent to the virtual negative space of the virtual 3D mold. At box 210, the method 200 comprises filling the physical negative space of the physical mold with a mold material to form the plug 102. At box 212, the method 200 comprises allowing the mold material forming the plug 102 to cure.

The method 200 shown in FIG. 7 may have additional steps. For example, the method 200 may include measuring a stoma surface geometry, wherein the virtual negative space includes a body geometry between the first end and the second end that is complementary to the stoma surface geometry. The method 200 may further include measuring a neck surface geometry, wherein the virtual negative space includes a face plate geometry complementary to the neck surface geometry. The minimum stoma diameter and the stoma length may be determined based on at least one of hand-collected detailed anatomic measurements, a 3D reformatted CT, a 3D reformatted MRI, and a surface 3D scan. Similarly, the stoma surface geometry and/or the face plate geometry may be determined based on at least one of a 3D reformatted CT, a 3D reformatted MRI, and a surface 3D scan.

FIG. 8 shows a physical mold 214 having a physical negative space 216 created according to the mold-based method 200. The physical mold 214 replicates in part the detailed anatomy of a patient's stoma and may be used to create a customized plug, such as plug 102 discussed above. The physical negative space 216 is roughly equivalent to the shape that the plug 102 will take. The physical mold 214 is manufactured to comply with FDA regulatory constraints. The physical mold 214 may comprise a medical grade photopolymer, such as an FDA cleared class I medical grade photopolymer. The physical mold 214 may be manufactured using additive manufacturing. That is, the virtual 3D mold described in step 208 of method 200 may be printed using any known 3D printing technique. In some instances, the physical mold 214 may undergo standard post-processing to, for example, remove undesirable ridges or artifacts from the printing process.

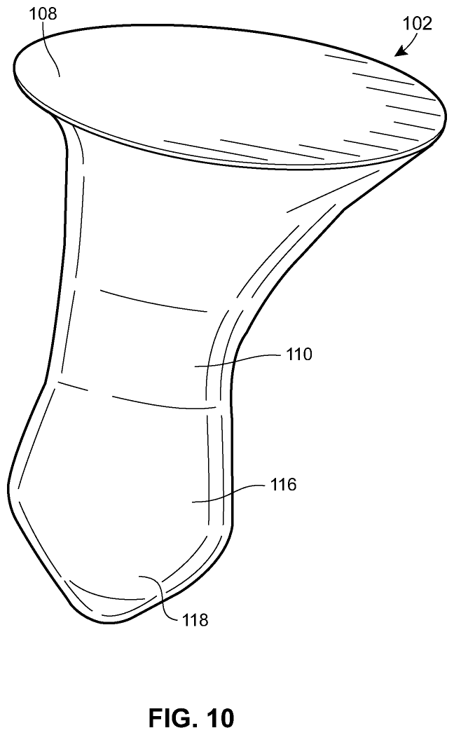

FIG. 9 shows the physical mold 214 of FIG. 8 after the physical negative space 216 has been filled with a mold material in order to create a plug such as plug 102. The mold material may be a medical grade silicone or another semi-rigid material approved for medical applications. After the mold material has cured, the customized plug 102 may be removed from the physical mold 214, as shown in FIG. 10. The customized plug 102 may undergo standard post-processing to remove undesirable ridges or artifacts created during the molding process.

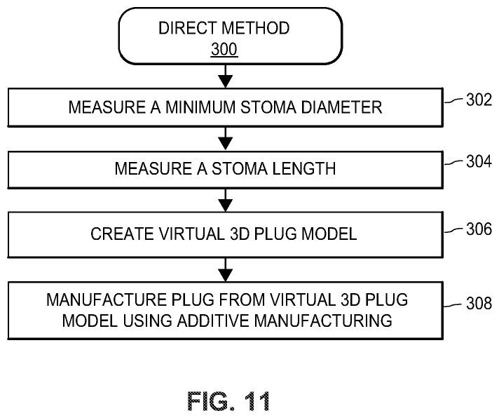

FIG. 11 depicts a direct method 300 of manufacturing a plug 102. At box 302, the method includes measuring a minimum stoma diameter. At box 304, the method 300 includes measuring a stoma length. At box 306, the method 300 includes creating a virtual 3D plug model, the virtual 3D plug model having a face plate having a face plate diameter greater than the minimum stoma diameter, and a body having a first end connected to the first plate and a second end, a shoulder connected at the second end, and a tapered tip extending from the shoulder, wherein the body has a length extending from the first end to the second end that is equal or substantially equal to the stoma length and a shoulder diameter that is greater than the minimum stoma diameter. At box 308, the method 300 includes manufacturing the plug 102 from the virtual 3D plug model using additive manufacturing. The plug 102 may be manufactured using a medical grade silicone or another semi-rigid material approved for medical applications. The customized plug 102 may undergo standard post-processing to remove undesirable ridges or artifacts created during the printing process.

The method 300 shown in FIG. 11 may have additional steps. For example, the method 300 may include measuring a stoma surface geometry, wherein the virtual negative space includes a body geometry between the first end and the second end that is complementary to the stoma surface geometry. The method 300 may further include measuring a neck surface geometry, wherein the virtual negative space includes a face plate geometry complementary to the neck surface geometry. The minimum stoma diameter and the stoma length may be determined based on at least one of hand-collected detailed anatomic measurements, a 3D reformatted CT, a 3D reformatted MRI, and a surface 3D scan. Similarly, the stoma surface geometry and/or the face plate geometry may be determined based on at least one of a 3D reformatted CT, a 3D reformatted MRI, and a surface 3D scan.

While specific embodiments have been described herein, variations may be made to the described embodiments that are still considered within the scope of the appended claims.

1-12. (canceled)

13. A mold-based method of manufacturing a tracheocutaneous fistula and tracheostomy plug, the method comprising:
measuring a minimum stoma diameter;
measuring a stoma length;
creating a virtual 3D mold for the plug having a virtual negative space for
a face plate having a face plate diameter greater than the minimum stoma diameter, and
a body having a first end connected to the first plate and a second end, a shoulder connected at the second end, and a tapered tip extending from the shoulder, wherein the body has a length extending from the first end to the second end that is equal or substantially equal to the stoma length and a shoulder diameter that is greater than the minimum stoma diameter;
manufacturing a physical mold having a physical negative space equivalent to the virtual negative space of the virtual 3D mold;
filling the physical negative space of the physical mold with a mold material to form the plug; and
allowing the mold material forming the plug to cure.

14. The mold-based method of claim 13, wherein the minimum stoma diameter and the stoma length are each determined based on at least one of hand-collected detailed anatomic measurements, a 3D reformatted CT, a 3D reformatted MRI, and a surface 3D scan.

15. The mold-based method of claim 13, wherein the physical mold comprises a medical grade photopolymer.

16. The mold-based method of claim 13, wherein the mold material is a medical grade silicone.

17. The mold-based method of claim 13, further comprising:
measuring a stoma surface geometry;
wherein the virtual negative space includes a body geometry between the first end and the second end that is complementary to the stoma surface geometry.

18. The mold-based method of claim 17, wherein the stoma surface geometry is determined based on at least one of a 3D reformatted CT, a 3D reformatted MRI, and a surface 3D scan.

19. The mold-based method of claim 13, further comprising:
measuring a neck surface geometry;
wherein the virtual negative space includes a face plate geometry complementary to the neck surface geometry.

20. The mold-based method of claim 19, wherein the face plate geometry is determined based on at least one of a 3D reformatted CT, a 3D reformatted MRI, and a surface 3D scan.

21. A direct method of manufacturing a tracheocutaneous fistula and tracheostomy plug, the method comprising:
measuring a minimum stoma diameter;
measuring a stoma length;
creating a virtual 3D plug model, the virtual 3D plug model having
a face plate having a face plate diameter greater than the minimum stoma diameter, and
a body having a first end connected to the first plate and a second end, a shoulder connected at the second end, and a tapered tip extending from the shoulder, wherein the body has a length extending from the first end to the second end that is equal or substantially equal to the stoma length and a shoulder diameter that is greater than the minimum stoma diameter;
manufacturing the plug from the virtual 3D plug model using additive manufacturing.

22. The direct method of claim 21, further comprising:
measuring a stoma surface geometry; and
measuring a neck surface geometry;
wherein the virtual 3D plug model further comprises a body geometry between the first end and the second end that is complementary to the stoma surface geometry and a face plate geometry complementary to the neck surface geometry.

23. The mold-based method of claim 13, wherein the body is a solid body.
